(19) United States
(12) Patent Application Publication
(10) Pub. No.: US 2026/0007653 A1
(43) Pub. Date: Jan. 8, 2026

(54) FORMULATIONS OF GLUCOCORTICOID RECEPTOR MODULATORS

(71) Applicant: Corcept Therapeutics Incorporated, Redwood City, CA (US)

(72) Inventors: Rick Bastin, Menlo Park, CA (US); Alaa Hosny, Nottingham (GB); Dolly Jacob, Nottingham (GB); Wu Lin, Nottingham (GB)

(21) Appl. No.: 19/240,547

(22) Filed: Jun. 17, 2025

Related U.S. Application Data
(62) Division of application No. 18/481,549, filed on Oct. 5, 2023, now Pat. No. 12,370,188.
(60) Provisional application No. 63/378,578, filed on Oct. 6, 2022.

(51) Int. Cl. A61K 31/4745; A61K 9/48; A61K 47/10; A61K 47/22

(52) U.S. Cl. CPC A61K 31/4745; A61K 9/4825; A61K 9/4858; A61K 9/4866; A61K 47/10; A61K 47/22

(57) ABSTRACT

The present invention provides formulations of (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone, and methods of making and using the same.

### CROSS-REFERENCE TO RELATED APPLICATIONS

This application is a continuation of U.S. Ser. No. 18/481,549, filed Oct. 5, 2023, which claims priority to U.S. Provisional Application No. 63/378,578, filed Oct. 6, 2022, which is incorporated by reference herein in its entirety for all purposes.

### BACKGROUND

In most species, including man, the physiological glucocorticoid is cortisol (hydrocortisone). Glucocorticoids are secreted in response to ACTH (corticotropin), which shows both circadian rhythm variation and elevations in response to stress and food. Cortisol levels are responsive within minutes to many physical and psychological stresses, including trauma, surgery, exercise, anxiety and depression. Cortisol is a steroid and acts by binding to an intracellular, glucocorticoid receptor (GR). A mineralocorticoid receptor (MR), also known as a type I glucocorticoid receptor (GR I), binds cortisol, and may also be activated by aldosterone in humans. Compositions including modulators of one or both of GR and MR may be used to treat a variety of diseases and disorders. In man, GR may be present in two forms: a ligand-binding GR-alpha of 777 amino acids; and, a GR-beta isoform which lacks the 50 carboxy terminal residues. Since these residues include the ligand binding domain, GR-beta is unable to bind the natural ligand, and is constitutively localized in the nucleus.

The biologic effects of cortisol, including those caused by hypercortisolemia, can be modulated at the GR level using receptor modulators, such as agonists, partial agonists and antagonists. Several different classes of agents are able to inhibit the physiologic effects of GR-agonist binding. These antagonists include compositions which, by binding to GR, inhibit the ability of an agonist to effectively bind to and/or activate the GR. One such known GR antagonist, mifepristone, has been found to be an effective anti-glucocorticoid agent in humans (Bertagna (1984) J. Clin. Endocrinol. Metab. 59:25). Mifepristone binds to the GR with high affinity, with a dissociation constant (Kd) of 10⁻⁹ M (Cadepond (1997) Annu. Rev. Med. 48:129).

In addition to cortisol, the biological effects of other steroids can be modulated at the GR level using receptor modulators, such as agonists, partial agonists and antagonists. When administered to subjects in need thereof, steroids can provide both intended therapeutic effects as well as negative side effects.

Amyotrophic lateral sclerosis (ALS) is a rare and devastating disease with insufficient treatment options. It is characterized by progressive degeneration of motor neurons in both the brain and the spinal cord leading to progressive muscle weakness, relentless disability and, generally, death within 3-5 years from symptom onset. Only a small percentage of patients survive more than 10 years. Fifty percent of patients die within 30 months of symptom onset; approximately 20% survive 5-10 years after onset (Riva N, Agosta F, Lunetts C, Filippi M, Quattrini A. 2016. Recent advances in amyotrophic lateral sclerosis. J. Neurol. 263:1241-1254). Respiratory failure is the most common cause of mortality in patients with ALS (Riva et al. 2016; Turner M R, Hardman O, Benatar M, Brooks B R, Chio A, de Carvalho M, et al. 2013. Controversies and priorities in amyotrophic lateral sclerosis. Lancet Neurol. 12:310-322). Familial ALS accounts for 10% of cases, with sporadic ALS accounting for the remaining 90% of cases (Van Damme P, Robberecht W, Van Den Bosch L. 2017. Modelling amyotrophic lateral sclerosis: progress and possibilities. Dis. Model. Mech. 10:537-549).

The compounds of U.S. Pat. No. 8,859,774 have demonstrated utility for treating this condition. What is needed are new forms of these compositions. Surprisingly, the present invention meets these and other needs.

### BRIEF SUMMARY OF THE INVENTION

In one embodiment, the present invention provides a composition comprising:

### Compound I, (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone

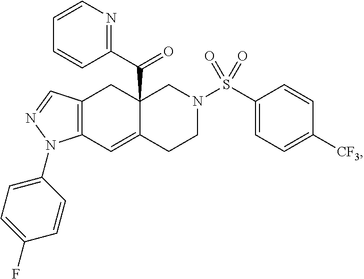 [SMILES: O=C(c1ccccn1)[C@]12Cc3cnn(-c4ccc(F)cc4)c3C=C1CCN(S(=O)(=O)c1ccc(C(F)(F)F)cc1)C2]

in an amount of from 1 to 25% (w/w); a cosolvent, in an amount of from 1 to 25% (w/w); and a surfactant, in an amount of from 50 to 90% (w/w).

In another embodiment, the present invention provides a unit dosage form for oral administration, consisting essentially of a capsule containing a composition of the present invention.

In another embodiment, the present invention provides a method of treating a disorder or condition through modulating a glucocorticoid receptor, comprising administering to a subject in need of such treatment, a therapeutically effective amount of a composition of the present invention, thereby treating the disorder or condition.

In another embodiment, the present invention provides a method of treating a disorder or condition through antagonizing a glucocorticoid receptor, comprising administering to a subject in need of such treatment, a therapeutically effective amount of a composition of the present invention, thereby treating the disorder or condition.

In another embodiment, the present invention provides a method of treating amyotrophic lateral sclerosis (ALS), comprising administering to a subject in need thereof, a therapeutically effective amount of a composition of the present invention, thereby treating ALS.

In another embodiment, the present invention provides a method of treating Alzheimer's disease, comprising administering to a subject in need thereof, a therapeutically effective amount of a composition of the present invention, thereby treating Alzheimer's.

In another embodiment, the present invention provides a method of treating Huntington's disease, comprising administering to a subject in need thereof, a therapeutically effective amount of a composition of the present invention, thereby treating Huntington's.

### DETAILED DESCRIPTION OF THE INVENTION

### I. General

Disclosed herein are formulations of Compound I, (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone:

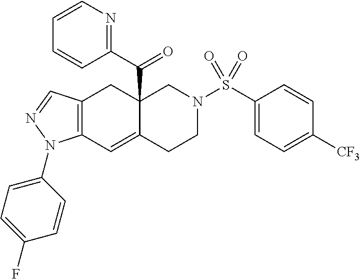 [SMILES: O=C(c1ccccn1)[C@]12Cc3cnn(-c4ccc(F)cc4)c3C=C1CCN(S(=O)(=O)c1ccc(C(F)(F)F)cc1)C2]

with a cosolvent and a surfactant.

### II. Definitions

“About” refers to plus or minus 5% of the specified value unless otherwise indicated.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier(s), diluent(s) or excipient(s) must be compatible with the other ingredients of the formulation and not deleterious to the recipient thereof.

“Cosolvent” refers to a component in a composition that improves the solubility and miscibility of other components in the composition.

“Surfactant” refers to any agent that alters the surface properties between two liquids, or between a liquid and a solid. Surfactants useful in the present invention include, but are not limited to, a non-ionic surfactant, a cationic surfactant, an anionic surfactant, an amphoteric surfactant, an ampholytic surfactant or salts thereof. A surfactant's hydrophilic/lipophilic balance (HLB) describes the surfactant's affinity toward water or oil (1-20, with 1 being lipophilic and 20 being hydrophilic). The HLB of a blend of two surfactants equals the weight fraction of surfactant A times its HLB value plus the weight fraction of surfactant B times its HLB value (weighted average). Anionic surfactants useful in the present invention include, but are not limited to soaps including alkali soaps, such as sodium, potassium and ammonium salts of aliphatic carboxylic acids, usually fatty acids, such as sodium stearate. Additional anionic surfactants include organic amine soaps such as organic amine salts of aliphatic carboxylic acids, usually fatty acids, such as triethanolamine stearate. Cationic surfactants useful in the present invention include, but are not limited to, amine salts such as octadecyl ammonium chloride and quarternary ammonium compounds such as benzalkonium chloride. Non-ionic surfactants useful in the present invention include but are not limited to polyoxyethylene castor oil derivatives, polysorbates, sorbitan esters, polyoxyethylene alkyl ethers, poloxamers and vitamin E derivatives. One of skill in the art will appreciate that other surfactants are useful in the present invention

“Pharmaceutically acceptable excipient” refers to a substance that aids the administration of an active agent to and absorption by a subject. Pharmaceutical excipients useful in the present invention include, but are not limited to, oils, fatty acid esters, cosolvents, surfactants, cosurfactants and antioxidants. One of skill in the art will recognize that other pharmaceutical excipients are useful in the present invention.

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Patient” or “subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, horse, and other non-mammalian animals. In some embodiments, the patient is human.

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by one skilled in the art using known techniques (see, e.g., Fridrun and Jones, Pharmaceutical Capsules, (2004), Lieberman, Pharmaceutical Dosage Forms (Disperse Systems vols. 1-3, 1992); Lloyd, The Art, Science and Technology of Pharmaceutical Compounding (1999); Pickar, Dosage Calculations (1999); and Remington: The Science and Practice of Pharmacy, 20^{th} Edition, 2003, Gennaro, Ed., Lippincott, Williams & Wilkins).

“Glucocorticoid receptor” (“GR”) refers to one of the family of intracellular receptors which specifically bind to cortisol and/or cortisol analogs such as dexamethasone (See, e.g., Turner & Muller, J. Mol. Endocrinol. Oct. 1, 2005 35 283-292). The glucocorticoid receptor is also referred to as the cortisol receptor. The term includes isoforms of GR, recombinant GR and mutated GR.

A cortisol receptor is a glucocorticoid receptor (GR), specifically the type II GR, which specifically binds cortisol and/or cortisol analogs such as dexamethasone (See, e.g., Turner & Muller, J. Mol. Endocrinol. Oct. 1, 2005 35 283-292).

“Mineralocorticoid receptor” (MR) refers to a type I glucocorticoid receptor (GR I), which is activated by aldosterone in humans.

“Glucocorticoid receptor modulator” (GRM) refers to any compound which modulates any biological response associated with the binding of a glucocorticoid receptor to an agonist. As used herein, with respect to a GRM, the glucocorticoid receptor may be GR, or both. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist, such as mifepristone, inhibits the agonist-induced increase in the activity of tyrosine aminotransferase (TAT) in HepG2 cells. TAT activity can be measured as outlined in the literature by A. Ali et al., J. Med. Chem., 2004, 47, 2441-2452.

“Glucocorticoid receptor antagonist” (GRA) refers to any compound which inhibits any biological response associated with the binding of a glucocorticoid receptor to an agonist. As used herein, with respect to a GRA, the glucocorticoid receptor may be GR, or both. Accordingly, GR antagonists can be identified by measuring the ability of a compound to inhibit the effect of dexamethasone. TAT activity can be measured as outlined in the literature by A. Ali et al., J. Med. Chem., 2004, 47, 2441-2452. An inhibitor is a compound with an IC₅₀ (half maximal inhibition concentration) of less than 10 micromolar.

“Modulate” and “modulating” are used in accordance with its plain ordinary meaning and refer to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Modulator” refers to a composition that increases or decreases the level of a target molecule or the function of a target molecule or the physical state of the target of the molecule.

“Antagonize’ and “antagonizing” refer to inhibiting the binding of an agonist at a receptor molecule or to inhibiting the signal produced by a receptor-agonist. A receptor antagonist inhibits or dampens agonist-mediated responses, such as gene expression.

“Antagonist” refers to a substance capable of detectably lowering expression or activity of a given gene or protein. The antagonist can inhibit expression or activity 10%, 20%, 30%, 40%, 50%, 60%, 70%, 80%, 90%, 100% or less in comparison to a control in the absence of the antagonist. In some embodiments, the inhibition is 1.5-fold, 2-fold, 3-fold, 4-fold, 5-fold, 10-fold, or more than the expression or activity in the absence of the antagonist.

“Inhibition”, “inhibits” and “inhibitor” refer to a compound that prohibits or a method of prohibiting, a specific action or function.

“Disorder” or “condition” refers to a state of being or health status of a patient or subject capable of being treated with the glucocorticoid receptor modulator of the present invention.

“Amyotrophic lateral sclerosis”, “ALS” and “Lou Gehrig's disease” refers to group of neurodegenerative diseases characterized by the loss of motor neurons in the ventral horns of the spinal cord and the cortical neurons that provide their afferent input. ALS can initially affect principally either the upper or lower motor neurons, but irrespective of the primary lesion area, with time, the disease acquires a symmetrical generalized nature (Mitsumoto, H. et al. Amyotrophic Lateral Sclerosis, In Contemporary Neurology Series 49, Philadelphia, F. A. Davis Company (1998)). Both sporadic and familial forms of ALS occur with familial ALS, usually autosomal dominant, representing about 10% of ALS (Dion P A et al., Nat. Rev. Genet. 10:769-782 (2009)). ALS symptoms typically appear earlier in familial cases, but the clinical courses of familial and sporadic forms are comparable. Several types of genetic mutations have been identified as causative for the development of familial ALS with approximately 20% of the familial cases caused by inherited mutations in the protein Cu/Zn superoxide dismutase (SOD1) that protects motor neurons from free radical damage (Rosen D R et al., Nature, 362:59-62 (1993)).

Unlike some forms of familial ALS, the specific the etiology of sporadic ALS remains elusive with different hypotheses proposed including glutamate-mediated excitotoxicity, impaired mitochondrial function, oxidative stress, neuroinflammation and aberrant protein aggregation (Dib M, Drugs, 63: 289-310 (2003); Strong M J, Pharmacology & Therapeutics, 98:379-414 (2003); Bruijn L I et al., Annu. Rev. Neurosci, 27:723-749 (2004); Dibernardo A B et al., Biochimica et Biophysica Acta, 1762:1139-1149 (2006)).

“A,” “an,” or “a(n)”, when used in reference to a group of substituents or “substituent group” herein, mean at least one. For example, where a compound is substituted with “an” alkyl or aryl, the compound is optionally substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### III. Compositions

The present invention provides pharmaceutically acceptable compositions of (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone (Compound I; see Example 1 of U.S. Pat. No. 8,859,774) which provide surprisingly improved solubility and stability of the composition. Compound I is practically insoluble in water and difficult to solubilize in most dosage forms that would be suitable for pharmaceutical use; routine methods have proven unsuccessful in providing pharmaceutically acceptable compositions of this compound. Compositions prepared by routine methods, even if able to solubilize small amounts of Compound I, have proven unstable with Compound I falling out of solution, or in which Compound I is found to be subject to rapid degradation, or incompatible with a pharmaceutically acceptable capsule. Surprisingly, the compositions disclosed herein overcome the previous problems of solubility and bioavailability, and provide pharmaceutically acceptable compositions with enhanced solubility and bioavailability, suitable for use in treating conditions and disorders amenable to treatment by administration of Compound I.

The compositions of the present invention are Type IV compositions according to the Lipid Formulation Classification System (LFCS). The LFCS identifies the characteristics of lipid systems (C. W. Pouton, Eur. J. Pharm. Sci., 11 (Suppl. 2) (2000), pp. S93-S98). As classified in the LFCS, Type I formulations are oils which require digestion, Type II formulations are water-insoluble self-emulsifying drug delivery systems (SEDDS), Type III systems are SEDDS or self-micro emulsifying drug delivery systems (SMEDDS) or self-nano emulsifying drug delivery systems (SNEDDS) which contain some water-soluble surfactants and/or co-solvents (Type IIIA) or a greater proportion of water soluble components (Type IIIB). Type IV systems represent formulations which contain predominantly hydrophilic surfactants and co-solvents that disperse and dissolve in aqueous media to provide a solution. A further description of the Lipid Formulation Classification System can also be found in FABAD J. Pharm. Sci., pages 55-64, 2013.

The present invention provides compositions of (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone (Compound I; CORT113176; dazucorilant; see Example 1 of U.S. Pat. No. 8,859,774). In some embodiments, the present invention provides a composition including Compound I having the structure:

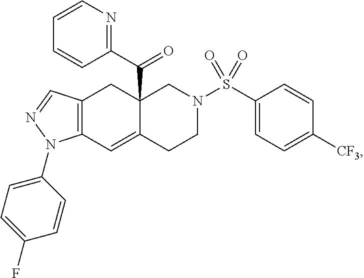 [SMILES: O=C(c1ccccn1)[C@]12Cc3cnn(-c4ccc(F)cc4)c3C=C1CCN(S(=O)(=O)c1ccc(C(F)(F)F)cc1)C2]

and a pharmaceutically acceptable excipient.

In some embodiments, the present invention provides a composition comprising: Compound I, (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone:

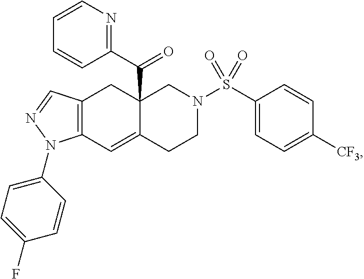 [SMILES: O=C(c1ccccn1)[C@]12Cc3cnn(-c4ccc(F)cc4)c3C=C1CCN(S(=O)(=O)c1ccc(C(F)(F)F)cc1)C2]

in an amount of from 1 to 25% (w/w); a cosolvent, in an amount of from 1 to 25% (w/w); and a surfactant, in an amount of from 50 to 90% (w/w).

### Compound I

Compound I can be present in the composition of the present invention in any suitable relative amount. For example, Compound I can be present in the composition of the present invention in an amount of from 1 to 50% (w/w), or from 1 to 25%, or from 5 to 40%, or from 10 to 30%, or from 10 to 25%, or from 13 to 22%, or from 10 to 20%, or from 15 to 20%, or from 13 to 17%, or from 14 to 16%, or from 18 to 22%, or from 18 to 20%, or from 18 to 22%, or from 19 to 21% (w/w). Representative amounts of Compound I in the compositions of the present invention include, but are not limited to, about 10% (w/w), or about 11, 12, 13, 14, 15, 16, 17, 18, 19, 20, 21, 22, 23, 24, or about 25% (w/w). Other amounts of Compound I useful in the compositions of the present invention include about 18.0% (w/w), or about 18.1, 18.2, 18.25, 18.3, 18.4, 18.5, 18.6, 18.7, 18.75, 18.8, 18.9, or about 19.0% (w/w).

In some embodiments, the compositions of the present invention include the composition wherein Compound I is present in an amount of from 1 to 25% (w/w). In some embodiments, the compositions of the present invention include the composition wherein Compound I is present in an amount of from 10 to 25% (w/w). In some embodiments, the compositions of the present invention include the composition wherein Compound I is present in an amount of from 15 to 20% (w/w). In some embodiments, the compositions of the present invention include the composition wherein Compound I is present in an amount of from 13 to 22% (w/w). In some embodiments, the compositions of the present invention include the composition wherein Compound I is present in an amount of from 15 to 20% (w/w). In some embodiments, the compositions of the present invention include the composition wherein Compound I is present in an amount of from 18 to 20% (w/w).

In some embodiments, the compositions of the present invention include the composition wherein Compound I is present in an amount of about 15% (w/w). In some embodiments, the compositions of the present invention include the composition wherein Compound I is present in an amount of about 18.75% (w/w). In some embodiments, the compositions of the present invention include the composition wherein Compound I is present in an amount of about 20% (w/w).

Compound I can be present in the composition of the present invention in any suitable absolute amount. For example, Compound I can be present in the composition of the present invention in an amount of from 1 to 500 mg, or 10 to 400 mg, or from 20 to 300 mg, or from 30 to 200 mg, or from 40 to 100 mg, or from 50 to 100 mg, or from 55 to 90 mg, or from 60 to 90 mg, or from 55 to 85 mg, or from 70 to 80 mg, or from 72 to 78 mg, or from 74 to 76 mg. Representative amounts of Compound I in the compositions of the present invention include, but are not limited to, about 10 mg, or 15, 20, 25, 30, 35, 40, 45, 50, 55, 60, 65, 70, 75, 80, 85, 90, 95, or about 100 mg. Other representative amounts of Compound I in the compositions of the present invention include, but are not limited to, about 55 mg, or 56, 57, 58, 59, 60, 61, 62, 63, 64, 65, 66, 67, 68, 69, 70, 71, 72, 73, 74, 75, 76, 77, 78, 79, 80, 81, 82, 83, 84, or about 85 mg. Other representative amounts of Compound I in the compositions of the present invention include, but are not limited to, about 50 mg, 75 mg, 100 mg, or about 150 mg.

In some embodiments, the compositions of the present invention include the composition wherein Compound I is present in an amount of from 50 to 150 mg. In some embodiments, the compositions of the present invention include the composition wherein Compound I is present in an amount of from 50 to 100 mg. In some embodiments, the compositions of the present invention include the composition wherein Compound I is present in an amount of from 55 to 85 mg. In some embodiments, the compositions of the present invention include the composition wherein Compound I is present in an amount of from 55 to 65 mg. In some embodiments, the compositions of the present invention include the composition wherein Compound I is present in an amount of from 70 to 80 mg. In some embodiments, the compositions of the present invention include the composition wherein Compound I is present in an amount of from 75 to 85 mg.

In some embodiments, the compositions of the present invention include the composition wherein Compound I is present in an amount of about 60 mg. In some embodiments, the compositions of the present invention include the composition wherein Compound I is present in an amount of about 75 mg. In some embodiments, the compositions of the present invention include the composition wherein Compound I is present in an amount of about 80 mg.

In some embodiments, the compositions of the present invention include the composition wherein Compound I is present in an amount of about 50 mg. In some embodiments, the compositions of the present invention include the composition wherein Compound I is present in an amount of about 75 mg. In some embodiments, the compositions of the present invention include the composition wherein Compound I is present in an amount of about 100 mg. In some embodiments, the compositions of the present invention include the composition wherein Compound I is present in an amount of about 125 mg. In some embodiments, the compositions of the present invention include the composition wherein Compound I is present in an amount of about 150 mg.

### Cosolvent

The cosolvent of the compositions of the present invention can include any suitable cosolvent. For example, the cosolvent in the compositions of the present invention can include, but are not limited to, polyethylene glycols (PEG), ethanol, propylene glycol, glycerol, diethylene glycol monoethyl ether, glycofurol, triacetin, N-methyl-2-pyrrolidone, dimethylacetamide, dimethylsulfoxide, propylene glycol esters, or combinations thereof.

In some embodiments, the compositions of the present invention include the composition wherein the cosolvent includes polyethylene glycols (PEG), ethanol, propylene glycol, glycerol, diethylene glycol monoethyl ether, glycofurol, triacetin, N-methyl-2-pyrrolidone, dimethylacetamide, dimethylsulfoxide, propylene glycol esters, or combinations thereof. In some embodiments, the compositions of the present invention include the composition wherein the cosolvent comprises polyethylene glycol (PEG).

When the cosolvent of the compositions of the present invention is polyethylene glycol (PEG), the PEG can have any suitable molecular weight that is liquid or semisolid at room temperature. For example, the PEG cosolvent in the compositions of the present invention can have a molecular weight of from 100 to 5000 Daltons, or from 100 to 2500 Daltons, or from 100 to 1000 Daltons, or from 100 to 750 Daltons, or from 200 to 600 Daltons, or from 300 to 500 Daltons. Representative molecular weights of the PEG cosolvent in the compositions of the present invention include, but are not limited to, about 220 Daltons, or 260, 310, 350, 400, 440, 480, 530, 570, 620, or about 660 Daltons. In some embodiments, the compositions of the present invention include the composition wherein the cosolvent comprises polyethylene glycol having a molecular weight of about 400 Daltons (PEG400).

The cosolvent can be present in the compositions of the present invention in any suitable amount. For example, the cosolvent can be present in the compositions of the present invention in an amount of from 1 to 90% (w/w), or from 5 to 25% (w/w), or from 2 to 20%, or from 3 to 15%, or from 4 to 10%, or from 5 to 8%, or from 6 to 7% (w/w). Representative amounts of the cosolvent in the compositions of the present invention include, but are not limited to, about 1% (w/w), or 2, 3, 4, 5, 6, 7, 8, 9, or about 10% (w/w). Other representative amounts of the cosolvent in the compositions of the present invention include, but are not limited to, about 6.1, 6.2, 6.3, 6.4, 6.5, 6.6, 6.7, 6.8, or about 6.9% (w/w).

In some embodiments, the compositions of the present invention include the composition wherein the cosolvent is present in an amount of from 1 to 25% (w/w). In some embodiments, the compositions of the present invention include the composition wherein the cosolvent comprises PEG400 in an amount of from 5 to 8% (w/w). In some embodiments, the compositions of the present invention include the composition wherein the cosolvent comprises PEG400 in an amount of from 6 to 7% (w/w). In some embodiments, the compositions of the present invention include the composition wherein the cosolvent comprises PEG400 in an amount of about 6.5% (w/w).

The cosolvent can be present in the composition of the present invention in any suitable absolute amount. For example, cosolvent can be present in the composition of the present invention in an amount of from 1 to 100 mg, or 5 to 50 mg, or from 10 to 40 mg, or from 15 to 35 mg, or from 20 to 30 mg, or from 22 to 28 mg, or from 24 to 28 mg, or from 25 to 27 mg. Representative amounts of cosolvent in the compositions of the present invention include, but are not limited to, about 15 mg, or 16, 17, 18, 19, 20, 21, 22, 23, 24, 25, 26, 27, 28, 29, 30, 31, 32, 33, 34, or about 35 mg.

In some embodiments, the compositions of the present invention include the composition wherein the cosolvent is present in an amount of from 20 to 30 mg. In some embodiments, the compositions of the present invention include the composition wherein the cosolvent is present in an amount of about 26 mg.

### Surfactant

The surfactant of the compositions of the present invention can include any suitable surfactant. The surfactants of the compositions of the present invention are generally hydrophilic non ionic surfactants, and generally have an HLB value of greater than 10, or greater than 12. Other non ionic surfactants useful in the compositions of the present invention have an HLB value greater than 4. For example, the surfactant of the compositions of the present invention includes, but is not limited to, Vitamin E polyethylene glycol succinate (Vitamin E TPGS), sorbitan monooleate, sorbitan monolaurate, polysorbate 80, polysorbate 20, Solutol HS 15, poloxamer 407 or poloxamer 168, Labrafil® M-1944CS, Labrafil M-2125CS, Labrasol® (Gattefosse, Saint-Priest, Lyon, France) Softigen® 767 (IOI Oleo GmbH, Germany), Gelucires (including Gelucire 44/14, 48/16 and 50/13), Kolliphor RH40, Kolliphor® EL (also known as cremophor), Kolliphor P188, Kolliphor® HS 15, Kolliphor® RH40 is Macrogolglycerol hydroxystearate (available from SIGMA-Aldrich, St. Louis, MO, USA), or combinations thereof.

In some embodiments, the compositions of the present invention include the composition wherein the surfactant includes Vitamin E polyethylene glycol succinate (Vitamin E TPGS), polysorbate, Solutol HS 15, poloxamer 407, Labrafil® M-1944CS, Labrafil M-2125CS, Labrasol®, Softigen® 767, Gelucire, Kolliphor, or combinations thereof. In some embodiments, the compositions of the present invention include the composition wherein the surfactant comprises Vitamin E polyethylene glycol succinate (Vitamin E TPGS). In some embodiments, the compositions of the present invention include the composition comprising PEG400 and Vitamin E TPGS.

The surfactant can be present in the compositions of the present invention in any suitable amount. For example, the surfactant can be present in the compositions of the present invention in an amount of from 10 to 95% (w/w), or from 25 to 95% (w/w), or from 50 to 95% (w/w), or from 50 to 90% (w/w), or from 60 to 90% (w/w), or from 65 to 85% (w/w), or from 70 to 80% (w/w), or from 72 to 78% (w/w), or from 72 to 76% (w/w), or from 74 to 76% (w/w), or from 76 to 80% (w/w). Representative amounts of the surfactant in the compositions of the present invention include, but are not limited to, about 50% (w/w), or 55, 60, 65, 70, 75, 80, 85, 90, or about 95% (w/w). Other representative amounts of the surfactant in the compositions of the present invention include, but are not limited to, about 71, or 72, 73, 74, 75, 76, 77, 78, or about 79% (w/w). Other representative amounts of the surfactant in the compositions of the present invention include, but are not limited to, about 73.1% (w/w), or 73.2, 73.25, 73.3, 73.4, 73.5, 73.6, 73.7, 73.75, 73.8, 73.9, 74.0, 74.1, 74.2, 72.25, 74.3, 74.4, 74.5, 74.6, 74.7, 74.75, 74.8, or about 74.9% (w/w). Other representative amounts of the surfactant in the compositions of the present invention include, but are not limited to, about 78.1% (w/w), or 78.2, 78.25, 78.3, 78.4, 78.5, 78.6, 78.7, 78.75, 78.8, or about 78.9% (w/w).

In some embodiments, the compositions of the present invention include the composition wherein the surfactant is present in an amount of from 10 to 90% (w/w). In some embodiments, the compositions of the present invention include the composition wherein the surfactant is present in an amount of from 50 to 90% (w/w). In some embodiments, the compositions of the present invention include the composition wherein the surfactant comprises Vitamin E polyethylene glycol succinate (Vitamin E TPGS) in an amount of from 70 to 80% (w/w). In some embodiments, the compositions of the present invention include the composition wherein the surfactant comprises Vitamin E polyethylene glycol succinate (Vitamin E TPGS) in an amount of from 72 to 78% (w/w). In some embodiments, the compositions of the present invention include the composition wherein the surfactant comprises Vitamin E polyethylene glycol succinate (Vitamin E TPGS) in an amount of from 74 to 76% (w/w).

In some embodiments, the compositions of the present invention include the composition wherein the surfactant comprises Vitamin E polyethylene glycol succinate (Vitamin E TPGS) in an amount of about 73.5% (w/w). In some embodiments, the compositions of the present invention include the composition wherein the surfactant comprises Vitamin E polyethylene glycol succinate (Vitamin E TPGS) in an amount of about 74.75% (w/w). In some embodiments, the compositions of the present invention include the composition wherein the surfactant comprises Vitamin E polyethylene glycol succinate (Vitamin E TPGS) in an amount of about 78.5% (w/w).

The surfactant can be present in the composition of the present invention in any suitable absolute amount. For example, the surfactant can be present in the composition of the present invention in an amount of from 100 to 1000 mg, or from 100 to 500 mg, or from 150 to 450 mg, or from 200 to 400 mg, or from 250 to 350 mg, or from 275 to 325 mg. Representative amounts of surfactant in the compositions of the present invention include, but are not limited to, about 250, or 255, 260, 265, 270, 275, 280, 285, 290, 295, 300, 305, 310, 315, 320, 325, 330, 335, 340, 345, or about 350 mg. Other representative amounts of surfactant in the compositions of the present invention include, but are not limited to, about 291 mg, or 292, 293, 294, 295, 296, 297, 298, 299, 300, 301, 302, 303, 304, 305, 306, 307, 308, 309, 310, 311, 312, 313, 314, 315, 316, 317, 318, or about 319 mg.

In some embodiments, the compositions of the present invention include the composition wherein the surfactant is present in an amount of from 275 to 325 mg. In some embodiments, the compositions of the present invention include the composition wherein the surfactant is present in an amount of about 294 mg. In some embodiments, the compositions of the present invention include the composition wherein the surfactant is present in an amount of about 299 mg. In some embodiments, the compositions of the present invention include the composition wherein the surfactant is present in an amount of about 314 mg.

### Combinations

In some embodiments, the compositions of the present invention include the composition including Compound I, PEG400, and Vitamin E TPGS. In some embodiments, the compositions of the present invention consisting essentially of the composition including Compound I, PEG400, and Vitamin E TPGS. In some embodiments, the compositions of the present invention include the composition consisting of Compound I, PEG400, and Vitamin E TPGS.

In some embodiments, the compositions of the present invention include the composition comprising: Compound I, in an amount of about 18.75% (w/w); PEG400, in an amount of about 6.5% (w/w); and Vitamin E TPGS, in an amount of about 74.75% (w/w). In some embodiments, the compositions of the present invention include the composition comprising: Compound I, in an amount of about 75 mg; PEG400, in an amount of about 26 mg; and Vitamin E TPGS, in an amount of about 299 mg. In some embodiments, the compositions of the present invention include the composition consisting of: Compound I, in an amount of 75 mg; PEG400, in an amount of 26 mg; and Vitamin E TPGS, in an amount of 299 mg.

In some embodiments, the compositions of the present invention include the composition comprising: Compound I, in an amount of about 50 mg; PEG400, in an amount of about 17 mg; and Vitamin E TPGS, in an amount of about 200 mg. In some embodiments, the compositions of the present invention include the composition consisting of: Compound I, in an amount of 50 mg; PEG400, in an amount of 17 mg; and Vitamin E TPGS, in an amount of 200 mg.

In some embodiments, the compositions of the present invention include the composition comprising: Compound I, in an amount of about 75 mg; PEG400, in an amount of about 26 mg; and Vitamin E TPGS, in an amount of about 299 mg. In some embodiments, the compositions of the present invention include the composition consisting of: Compound I, in an amount of 75 mg; PEG400, in an amount of 26 mg; and Vitamin E TPGS, in an amount of 299 mg.

In some embodiments, the compositions of the present invention include the composition comprising: Compound I, in an amount of about 100 mg; PEG400, in an amount of about 35 mg; and Vitamin E TPGS, in an amount of about 399 mg. In some embodiments, the compositions of the present invention include the composition consisting of: Compound I, in an amount of 100 mg; PEG400, in an amount of 35 mg; and Vitamin E TPGS, in an amount of 399 mg.

In some embodiments, the compositions of the present invention include the composition comprising: Compound I, in an amount of about 125 mg; PEG400, in an amount of about 43 mg; and Vitamin E TPGS, in an amount of about 498 mg. In some embodiments, the compositions of the present invention include the composition consisting of: Compound I, in an amount of 125 mg; PEG400, in an amount of 43 mg; and Vitamin E TPGS, in an amount of 498 mg.

In some embodiments, the compositions of the present invention include the composition comprising: Compound I, in an amount of about 150 mg; PEG400, in an amount of about 52 mg; and Vitamin E TPGS, in an amount of about 598 mg. In some embodiments, the compositions of the present invention include the composition consisting of: Compound I, in an amount of 150 mg; PEG400, in an amount of 52 mg; and Vitamin E TPGS, in an amount of 598 mg.

In some embodiments, the compositions of the present invention include the composition comprising: Compound I, in an amount of about 15% (w/w); PEG400, in an amount of about 6.5% (w/w); and Vitamin E TPGS, in an amount of about 78.5% (w/w). In some embodiments, the compositions of the present invention include the composition comprising: Compound I, in an amount of about 60 mg; PEG400, in an amount of about 26 mg; and Vitamin E TPGS, in an amount of about 314 mg. In some embodiments, the compositions of the present invention include the composition consisting of: Compound I, in an amount of about 60 mg; PEG400, in an amount of about 26 mg; and Vitamin E TPGS, in an amount of about 314 mg. In some embodiments, the compositions of the present invention include the composition consisting of: Compound I, in an amount of 60 mg; PEG400, in an amount of 26 mg; and Vitamin E TPGS, in an amount of 314 mg.

In some embodiments, the compositions of the present invention include the composition comprising: Compound I, in an amount of about 20.0% (w/w); PEG400, in an amount of about 6.5% (w/w); and Vitamin E TPGS, in an amount of about 73.5% (w/w). In some embodiments, the compositions of the present invention include the composition comprising: Compound I, in an amount of about 80 mg; PEG400, in an amount of about 26 mg; and Vitamin E TPGS, in an amount of about 294 mg. In some embodiments, the compositions of the present invention include the composition consisting of: Compound I, in an amount of about 80 mg; PEG400, in an amount of about 26 mg; and Vitamin E TPGS, in an amount of about 294 mg. In some embodiments, the compositions of the present invention include the composition consisting of: Compound I, in an amount of 80 mg; PEG400, in an amount of 26 mg; and Vitamin E TPGS, in an amount of 294 mg.

The compositions of the present invention can be prepared and administered in a wide variety of oral dosage forms, including but not limited to, liquid filled capsules or solution products for oral or enteral adminsitration. Other oral preparations include tablets, pills, powder, dragees, capsules, slurries, suspensions, etc., suitable for ingestion by the patient. Accordingly, the present invention also provides pharmaceutical compositions including one or more pharmaceutically acceptable carriers and/or excipients and either a compound, or a pharmaceutically acceptable salt of a compound.

For preparing compositions from Compound I, pharmaceutically acceptable carriers can be either solid, semisolid or liquid. A solid carrier can be one or more substances, which may also act as solubilizers, emulsifier, dispersing agents, antioxidants, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

Additionally, the carriers or excipients used in the pharmaceutical compositions of this invention are commercially-available. By way of further illustration, conventional formulation techniques are described in Remington: The Science and Practice of Pharmacy, 20th Edition, Lippincott Williams & White, Baltimore, Md. (2000); and H. C. Ansel et al., Pharmaceutical Dosage Forms and Drug Delivery Systems, 7th Edition, Lippincott Williams & White, Baltimore, Md. (1999).

The pharmaceutical preparation can be prepared in a unit dosage form. In such form the preparation is subdivided into unit doses containing appropriate quantities of the active component. The unit dosage form can be a packaged preparation, the package containing discrete quantities of preparation, such as packeted capsules or solutions in bottles, vials or blister packs

The composition of the present invention can be in a unit dosage form. The unit dosage form of the compositions of the present invention can be of any suitable amount. For example, the compositions of the present invention can be in a unit dosage form of from 100 to 1000 mg, or from 200 to 750 mg, or from 250 to 750 mg, of rom 300 to 500 mg, or from 350 to 450 mg, or from 375 to 425 mg. Representative unit dosage form amounts for the compositions of the present invention include, but are not limited to, 100, 200, 250, 300, 350, 400, 450, 500, 550, 600, 650, 700, or about 750 mg. In some embodiments, the compositions of the present invention include the composition in a unit dosage form of 400 mg. In some embodiments, the compositions of the present invention include the composition in a unit dosage form of 267 mg. In some embodiments, the compositions of the present invention include the composition in a unit dosage form of 533 mg. In some embodiments, the compositions of the present invention include the composition in a unit dosage form of 667 mg. In some embodiments, the compositions of the present invention include the composition in a unit dosage form of 800 mg.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, GR and/or MR modulator and disease or condition treated.

Single or multiple administrations of the compositions can be administered depending on the dosage and frequency as required and tolerated by the patient. The compounds should provide a sufficient quantity of active agent to effectively treat the disease state. Thus, in some embodiments, the pharmaceutical formulations for oral administration of the compound is in a daily amount of between about 0.5 to about 30 mg per kilogram of body weight per day. In some embodiments, dosages can be from about 1 mg to about 20 mg per kg of body weight per patient per day are used. Actual methods for preparing parenterally administrable formulations will be known or apparent to those skilled in the art and are described in more detail in such publications as Remington's, supra. See also Nieman, In “Receptor Mediated Antisteroid Action,” Agarwal, et al., eds., De Gruyter, New York (1987).

In some embodiments, the pharmaceutical formulations for oral administration of the compound are in a daily amount of between about 50 mg and about 300 mg of Compound I. In some embodiments, the daily amount of Compound I is about 50 mg, about 75 mg, about 100 mg, about 150 mg, or about 300 mg. In some embodiments, the daily amount of Compound I is provided in a single unit dosage form. In some embodiments, the daily amount of Compound I is provided in two, three, or four unit dosage forms each comprising 50 mg, 75 mg, 100 mg, or 150 mg of Compound I. In some embodiments, the daily amount of Compound I is provided in two, three, or four unit dosage forms each comprising 50 mg, 75 mg, or 100 mg of Compound I. In some embodiments, the daily amount is 75 mg Compound I, provided as one unit dosage form comprising 75 mg of Compound I. In some embodiments, the daily amount is 100 mg Compound I, provided as one unit dosage form comprising 100 mg of Compound I. In some embodiments, the daily amount is 100 mg Compound I, provided as two unit dosage forms each comprising 50 mg of Compound I. In some embodiments, the daily amount is 150 mg Compound I, provided as one unit dosage form comprising 150 mg of Compound I. In some embodiments, the daily amount is 150 mg Compound I, provided as two unit dosage forms each comprising 75 mg of Compound I. In some embodiments, the daily amount is 150 mg Compound I, provided as three unit dosage forms each comprising 50 mg of Compound I. In some embodiments, the daily amount is 300 mg Compound I, provided as two unit dosage forms each comprising 150 mg of Compound I. In some embodiments, the daily amount is 300 mg Compound I, provided as three unit dosage forms each comprising 100 mg of Compound I. In some embodiments, the daily amount is 300 mg Compound I, provided as four unit dosage forms each comprising 75 mg of Compound I.

Compound I described herein can be used in combination with other active agents known to be useful in modulating a glucocorticoid receptor, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another.

After a composition of the present invention has been formulated in one or more acceptable carriers, it can be placed in an appropriate container and labeled for treatment of an indicated condition. For administration of Compound I, such labeling would include, e.g., instructions concerning the amount, frequency and method of administration.

In some embodiments, the present invention provides a unit dosage form for oral administration, consisting essentially of a capsule containing a composition of the present invention. The capsule can be any suitable type of capsule. For example, the capsule can be a softgel capsule or a hardgel capsule. In some embodiments, the unit dosage form for oral administration of the present invention is the unit dosage form wherein the capsule is a softgel capsule or a hardgel capsule. In some embodiments, the unit dosage form for oral administration of the present invention is the unit dosage form wherein the capsule is a softgel capsule. In some embodiments, the unit dosage form for oral administration of the present invention is the unit dosage form wherein the capsule is a hardgel capsule.

In some embodiments, the unit dosage form for oral administration of the present invention is the unit dosage form consisting essentially of a softgel capsule containing the composition consisting of: Compound I, in an amount of 75 mg; polyethylene glycol having a molecular weight of about 400 Daltons (PEG400), in an amount of 26 mg; and Vitamin E polyethylene glycol succinate (Vitamin E TPGS), in an amount of 299 mg.

In some embodiments, the unit dosage form for oral administration of the present invention is the unit dosage form consisting essentially of a hardgel capsule containing the composition consisting of: Compound I, in an amount of 75 mg; polyethylene glycol having a molecular weight of about 400 Daltons (PEG400), in an amount of 26 mg; and Vitamin E polyethylene glycol succinate (Vitamin E TPGS), in an amount of 299 mg.

In some embodiments, the unit dosage form for oral administration of the present invention is the unit dosage form consisting essentially of a hardgel capsule containing the composition consisting of: Compound I, in an amount of 50 mg; polyethylene glycol having a molecular weight of about 400 Daltons (PEG400), in an amount of 17 mg; and Vitamin E polyethylene glycol succinate (Vitamin E TPGS), in an amount of 199 mg.

In some embodiments, the unit dosage form for oral administration of the present invention is the unit dosage form consisting essentially of a hardgel capsule containing the composition consisting of: Compound I, in an amount of 100 mg; polyethylene glycol having a molecular weight of about 400 Daltons (PEG400), in an amount of 35 mg; and Vitamin E polyethylene glycol succinate (Vitamin E TPGS), in an amount of 399 mg.

In some embodiments, the unit dosage form for oral administration of the present invention is the unit dosage form consisting essentially of a hardgel capsule containing the composition consisting of: Compound I, in an amount of 150 mg; polyethylene glycol having a molecular weight of about 400 Daltons (PEG400), in an amount of 52 mg; and Vitamin E polyethylene glycol succinate (Vitamin E TPGS), in an amount of 598 mg.

### IV. Methods of Use

In some embodiments, the present invention provides a method of treating a disorder or condition through modulating a glucocorticoid receptor, comprising administering to a subject in need of such treatment, a therapeutically effective amount of a composition of the present invention, thereby treating the disorder or condition.

In some embodiments, the present invention provides a method of treating a disorder or condition through antagonizing a glucocorticoid receptor, comprising administering to a subject in need of such treatment, a therapeutically effective amount of a composition of the present invention, thereby treating the disorder or condition.

In some embodiments, the present invention provides methods of modulating glucocorticoid receptor activity using the techniques described herein. In some embodiments, the method includes contacting a GR with an effective amount of a composition or Compound I of the present invention, and detecting a change in GR activity.

In some embodiments, the present invention provides methods of modulating glucocorticoid receptor activity using the techniques described herein. In some embodiments, the method includes contacting a GR or both with an effective amount of a composition or Compound I of the present invention, and detecting a change in GR activity, MR activity, or both.

In some embodiments, the glucocorticoid receptor modulator is an antagonist of GR activity or MR activity, or both GR and MR activity (also referred to herein as “a glucocorticoid receptor antagonist”). A glucocorticoid receptor antagonist, as used herein, refers to any composition or compound which partially or completely inhibits (antagonizes) the binding of a glucocorticoid receptor agonist (e.g. cortisol, aldosterone, and synthetic or natural cortisol or aldosterone analogs) to a GR, thereby inhibiting any biological response associated with the binding of a GR, to the agonist.

In some embodiments, the glucocorticoid receptor modulator is a specific glucocorticoid receptor antagonist. As used herein, a specific glucocorticoid receptor antagonist refers to a composition or compound which inhibits any biological response associated with the binding of a GR to an agonist by preferentially binding to the GR rather than another nuclear receptor (NR). In some embodiments, the specific glucocorticoid receptor antagonist binds preferentially to GR rather than the androgen receptor (AR), estrogen receptor (ER) or progesterone receptor (PR). In some embodiments, the specific glucocorticoid receptor antagonist binds preferentially to GR rather than the progesterone receptor (PR). In some embodiments, the specific glucocorticoid antagonist binds preferentially to GR rather than to the androgen receptor (AR). In some embodiments, the specific glucocorticoid antagonist binds preferentially to GR rather than to the estrogen receptor (ER).

In some embodiments, the specific glucocorticoid receptor antagonist binds to the GR with an association constant (Kd) that is at least 10-fold less than the Kd for AR or PR. In some embodiments, the specific glucocorticoid receptor antagonist binds to the GR with an association constant (Kd) that is at least 100-fold less than the Kd for AR or PR. In some embodiments, the specific glucocorticoid receptor antagonist binds to the GR with an association constant (Kd) that is at least 1000-fold less than the Kd for AR, PR or ER.

In some embodiments, the present invention provides a method of treating amyotrophic lateral sclerosis (ALS), comprising administering to a subject in need thereof, a therapeutically effective amount of a composition of the present invention, thereby treating ALS.

Amyotrophic lateral sclerosis (ALS; also known as Lou Gehrig's disease) is a progressive neuromuscular condition characterized by weakness, muscle wasting, fasciculations, and increased reflexes. Approximately 30,000 Americans are currently afflicted with the disease. The annual incidence rate is one to two cases per 100,000. The disease is most commonly diagnosed in middle age and affects more men than women. ALS is characterized by adult-onset, idiopathic, progressive degeneration of spinal anterior horn cells and upper and lower motor neurons resulting in progressive muscle weakness, wasting, and fasciculations. Atrophy of the spinal anterior horn cells and replacement of the large motor neurons by fibrous astrocytes (gliosis) causes the affected anterior and lateral columns of the spinal cord to become hard, hence the term “lateral sclerosis.” Typical signs and symptoms of ALS include muscle weakness, muscle wasting (atrophy), muscle fasciculations, muscle spasticity, slowness of movement, poor balance, incoordination, alterations in vocal quality, dysarthria, dysphagia, incomplete eye closure, drooling, pseudobulbar affect, and premature death.

Up to 10% of ALS is familial, usually autosomal dominant. Several causative genes are known and, of these, mutant superoxide dismutase 1 (SOD) and mutant C9orf72 (i.e., a G₄C₂ hexanucleotide repeat in the C9orf72 gene) are the most frequently found among familial ALS (fALS) and sporadic ALS (sALS). Several other genes are known to be causative of classical ALS, although these account for a lower percentage of cases than does mutant SOD1; these genes include mutant FUS (fused in sarcoma), mutant TARDBP gene leading to modifications of the TAR DNA binding protein 43 (TDP-43), and optineurin.

The clinical presentation varies, depending on the area of the nervous system that is damaged and progression of the pathologic changes. The classic presentation of ALS is insidious, progressive, asymmetric muscular weakness and atrophy along with neurologic signs, particularly fasciculations and hyperreflexia. It usually presents with problems in dexterity or gait resulting from muscle weakness. Difficulty in speaking or swallowing is the initial symptom in the bulbar form of the disease. Over a period of months or years, patients with ALS develop severe, progressive muscular weakness and other symptoms caused by loss of function in both upper and lower motor neurons. Sphincter control, sensory function, intellectual abilities and skin integrity are preserved. Patients become completely disabled, often requiring ventilatory support and gastrostomy. Death usually occurs within five years of diagnosis and is attributed to respiratory failure or cachexia. The diagnosis of ALS is clinical, based on the characteristic signs of progressive weakness, atrophy, fasciculations and hyperreflexia affecting several regions of the body. The early differential diagnosis may include musculoskeletal, neurologic or systemic conditions. The etiology of the disease is unknown. Current management involves aggressive, individualized alleviation of symptoms and complications.

Examples of disorders or conditions suitable for use with present invention include, but are not limited to, neurodegeneration, Alzheimer's disease, Huntingtons's disease, Parkinson's disease, cognition enhancement, mild cognitive impairment, psychosis, or dementia. In some embodiments, the present invention provides a method of treating Alzheimer's disease, comprising administering to a subject in need thereof, a therapeutically effective amount of a composition of the present invention, thereby treating Alzheimer's. In some embodiments, the present invention provides a method of treating Huntington's disease, comprising administering to a subject in need thereof, a therapeutically effective amount of a composition of the present invention, thereby treating Huntington's.

### V. Examples

### Example 1. Preparation of Compound I

Compound I can be prepared as described in U.S. Pat. No. 8,859,774 (incorporated herein in its entirety for all purposes), Example 1, (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone:

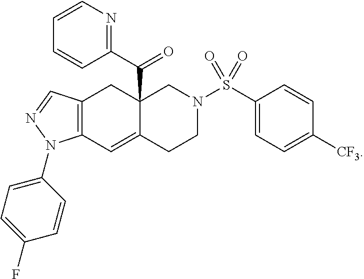 [SMILES: O=C(c1ccccn1)[C@]12Cc3cnn(-c4ccc(F)cc4)c3C=C1CCN(S(=O)(=O)c1ccc(C(F)(F)F)cc1)C2]

### Example 2. Preparation of Type IV Formulations of Compound I in a Softgel Capsule

Type IV formulations in softgel capsules were prepared according to the following method:
Step 1: The Vitamin E TPGS was preheated to a temperature of 65° C. until molten in a suitable oven and 74.75 g dispensed into a suitable mixing container. While maintaining a temperature of 40° C. and stirring, polyethylene glycol (6.5 g) and Compound I (18.75 g) were added to the container and the contents mixed until a visually homogeneous solution was formed. The resultant solution (fill matrix) was then deaerated, blanketed with nitrogen, and stored until encapsulation, and stored at 45-55° C. until encapsulation.
Step 2: The gel mass was prepared using gelatin, purified water, and sorbitol special glycerin blend. The gel mass was then color converted by adding titanium dioxide and red iron oxide.
Step 3: The fill matrix was encapsulated into softgel capsules.
Step 4: The capsules were dried and hardened in tumble dryers and drying chambers.
Step 5: The capsules are bulk packaged in polyethylene-lined corrugated shipping cartons.
Step 6: The capsules were packed into their final packaging configuration such as bottles or blisters.

TABLE 1
Type IV Formulation of Compound I
in a Softgel Capsule, 75 mg Strength

| | | | |
|---|---|---|---|
| | | mg per | |
| Component | % w/w | capsule | Function |

Capsule Fill Matrix

| | | | |
|---|---|---|---|
| Compound I | 18.75 | 75.0 | Active ingredient |
| Polyethylene Glycol 400 | 6.50 | 26.0 | Solubilizer & dispersion |
| | | | agent |
| Vitamin E TPGS | 74.75 | 299.0 | Solubilizer & surfactant |
| Total in capsule fill: | 100.0 | 400.0 | |

Capsule Shell

| | | | |
|---|---|---|---|
| Gelatin | 63.24 | 171.43 | Capsule shell |
| Sorbitol special glycerin | 36.14 | 97.96 | Shell plasticizer |
| blend (50:50 sorbitol | | | |
| solution/glycerin) | | | |
| Titanium dioxide | 0.49 | 1.32 | Colorant |
| Iron oxide, red | 0.14 | 0.38 | Colorant |
| Total in capsule shell: | 100.00 | 271.09 | |

TABLE 2
Type IV Formulation of Compound I, Fill matrix 60 mg Strength

| | | mg per | |
|---|---|---|---|
| Component | % w/w | capsule | Function |
| Compound I | 15.0 | 60.0 | Active ingredient |
| Polyethylene Glycol 400 | 6.50 | 26.0 | Solubilizer & dispersion |
| | | | agent |
| Vitamin E TPGS | 78.5 | 314.0 | Solubilizer & surfactant |
| Total in capsule fill: | 100.0 | 400.0 | |

TABLE 3
Type IV Formulation of Compound I, Fill matrix 80 mg Strength

| | | mg per | |
|---|---|---|---|
| Component | % w/w | capsule | Function |
| Compound I | 20.0 | 80.0 | Active ingredient |
| Polyethylene Glycol 400 | 6.50 | 26.0 | Solubilizer & dispersion |
| | | | agent |
| Vitamin E TPGS | 73.5 | 294 | Solubilizer & surfactant |
| Total in capsule fill: | 100.0 | 400.0 | |

### Example 3. Preparation of Type IV Formulation of Compound I in a Hardgel Capsule, 150 mg Strength

A Type IV formulation in hardgel capsules was prepared according to the following method:
Step 1: The Vitamin E TPGS was preheated to a temperature of 65° C. until molten in a suitable oven and 448.5 g was dispensed into a suitable mixing container. While maintaining a temperature of 45-55° C. and stirring, polyethylene glycol (39.0 g) and Compound I (112.5 g) were added to the container and the contents mixed until a visually homogeneous solution was formed. The resultant solution (fill matrix) was then stored at 45-55° C. until encapsulation.
Step 2: The molten fill matrix (800 mg) was encapsulated into size 00el hard gelatin capsules.
Step 3: The capsules were banded using a solution of 25% w/w gelatin. The capsules were then held on drying racks on the capsule banding equipment until dried and sealed.
Step 4: The capsules were packed into their final packaging configuration such as bottles or blisters.

TABLE 4
Type IV Formulation of Compound I, Fill
Matrix & Hardgel Capsule, 150 mg Strength

| | | | |
|---|---|---|---|
| | | mg per | |
| Component | % w/w | capsule | Function |

Capsule Fill Matrix

| | | | |
|---|---|---|---|
| Compound I | 18.75 | 150.0 | Active ingredient |
| Polyethylene Glycol 400 | 6.50 | 52.0 | Solubilizer & dispersion |
| | | | agent |
| Vitamin E TPGS | 74.75 | 598.0 | Solubilizer & surfactant |
| Total in capsule fill: | 100.0 | 800.0 | |

Capsule Shell

| | | |
|---|---|---|
| Swedish Orange capsule | 1 unit | Capsule shell |
| shell size 00el | | |
| Gelatin banding¹ | q.s | |

¹Applied as a 25% gelatin solution. Water removed during the drying process.

### Example 4. Stability Testing of Type III and IV Fill Matrices of Compound I

The Type IV fill matrix was prepared as described above, using the 18.75% w/w Compound I fill matrix shown in Table 1, and were tested under various accelerated stability conditions and compared against Type III fill matrices. For water spiked samples of Type IV formulations, 5 g water was stirred into the solution. The solution was then filled into vials while still molten and stored for in vial stability testing.

Samples of Type III formulations were prepared according to known methods using the w/w percentages in Table 5. For example, the Kolliphor ELP and Masester E8120 were preheated in a suitable oven to a temperature of 65° C. until molten. Caproyl 90 (32.24 g), Masester E8120 (24.83 g) and Kolliphor ELP (24.18 g) were added to a suitable mixing container and stirred until homogenous while maintaining a temperature of 40° C. Compound I (18.75 g) was added and mixed until dissolved and a fully homogenous solution was formed. For samples containing antioxidants, the required amount of antioxidant was added (0.167 g of dl-alpha tocopherol or 0.02 g of BHT) and the levels of Caproyl 90, Masester E8120 and Kolliphor ELP proportionally reduced to compensate for the antioxidant present. The solution is stirred until all materials have dissolved and the solution was homogenous. For water spiked samples, 5 g water was stirred into the solution.

TABLE 5
Type III Fill Matrix Formulation

| Component | Description/ | Percentage |
|---|---|---|
| (Chemical Name) | Tradename | Formula (% w/w) |
| Compound I | Drug substance | 18.75% |
| Propylene glycol | Capryol 90 ® | 32.24% |
| monocaprylate (Type II) | | |
| Glyceryl caprylate/caproate | Masester E8120 ® | 24.83% |
| Polyoxy1-35 castor oil | Kolliphor ELP ® | 24.18% |
| | Total | 100.00% |

The stability of the Type IV capsule fill matrix samples was assessed as a solution in closed glass screw capped vials stored under accelerated conditions and compared to the alternative Type III formulation with or without antioxidants. For example, a 2 g quantity of each solution type were filled into 4 mL amber glass vials, the headspace nitrogen purged and the vials capped. The samples were placed in stability chambers at 30° C./65% RH and 40° C./75% RH. The assay and levels of impurities were assessed over a period of up to 12 weeks using a suitable HPLC method.

For the antioxidants in the Type III formulations either butylated hydroxytoluene (BHT) at a typical usage level of 0.02% w/w or Vitamin E (dl-alpha tocopherol) at 0.16% w/w were added with a further sample without antioxidant being evaluated. Some vials were spiked with 5% water (wet samples) to encourage degradation as it is known that moisture can migrate from capsule shells into the fill matrix during manufacture or storage. Samples that were not spiked with water (dry samples) were also assessed. For the Type IV formulation an antioxidant was not added but both wet and dry samples were assessed. Samples were stored at 40° C./75% RH and 30° C./65% RH for up to twelve weeks and the assay and total impurities determined. The results are shown in Table 6, Table 7, Table 8, and Table 9.

TABLE 6
Assay Data for Capsule Fill Matrices Stored at 30° C./60% RH

| | | | Assay (percent) after Storage | | | |
|---|---|---|---|---|---|---|
| Formu- | | Water | at 40° C./75% RH | | | |
| lation | Antioxidant | Spiked | T = 0 | T = 4 w | T = 8 w | T = 12 w |
| Type III | Vitamin E | No | 100.7 | 98.8 | 98.8 | NA |
| | Vitamin E | Yes | 101.9 | 98.4 | 96.2 | NA |
| Type III | BHT | No | 101.1 | 98.9 | 98.8 | NA |
| | BHT | Yes | 101.3 | 97.9 | 97.0 | NA |
| Type III | None | No | 101.1 | 99.0 | 98.6 | NA |
| | None | Yes | 102.7 | 97.8 | 96.0 | NA |
| Type IV | None | No | 100.8 | 100.5 | 99.5 | 98.7 |
| | None | Yes | 102.8 | 106.3 | 101.7 | 103.4 |

NA - Not assessed

TABLE 7
Assay Data for Capsule Fill Matrices Stored at 40° C./75% RH

| | | | Assay (percent) after Storage | | | |
|---|---|---|---|---|---|---|
| Formu- | | Water | at 40° C./75% RH | | | |
| lation | Antioxidant | Spiked | T = 0 | T = 4 w | T = 8 w | T = 12 w |
| Type III | Vitamin E | No | 100.7 | 98.0 | 97.8 | NA |
| | Vitamin E | Yes | 101.9 | 94.9 | 89.6 | NA |
| Type III | BHT | No | 101.1 | 97.8 | 97.1 | NA |
| | BHT | Yes | 101.3 | 94.2 | 89.7 | NA |
| Type III | None | No | 101.1 | 98.0 | 97.6 | NA |
| | None | Yes | 102.7 | 94.7 | 89.2 | NA |
| Type IV | None | No | 100.8 | 102.8 | 99.4 | 98.3 |
| | None | Yes | 102.8 | 106.1 | 101.7 | 98.5 |

NA - Not assessed

TABLE 8
Impurity Data for Capsule Fill Matrices Stored at 30° C./65% RH

| | | | Percentage of Total Impurities | | | |
|---|---|---|---|---|---|---|
| | | | after Storage at 30° C./ | | | |
| Formu- | | Water | 65% RH (% area) | | | |
| lation | Antioxidant | Spiked | T = 0 | T = 4 w | T = 8 w | T = 12 w |
| Type III | Vitamin E | No | 0.50 | 0.54 | 0.69 | NA |
| | Vitamin E | Yes | 0.50 | 1.05 | 1.28 | NA |
| Type III | BHT | No | 0.60 | 0.71 | 0.84 | NA |
| | BHT | Yes | 0.70 | 1.11 | 1.71 | NA |
| Type III | None | No | 0.60 | 0.64 | 0.77 | NA |
| | None | Yes | 0.60 | 1.09 | 1.69 | NA |
| Type IV | None | No | 0.50 | 0.45 | 0.48 | 0.48 |
| | None | Yes | 0.40 | 0.46 | 0.49 | 0.49 |

NA = Not assessed

TABLE 9
Impurity Data for Capsule Fill Matrices Stored at 40° C./75% RH

| | | | Percentage of Total Impurities | | | |
|---|---|---|---|---|---|---|
| | | | after Storage at 40° C./ | | | |
| Formu- | | Water | 75% RH (% area) | | | |
| lation | Antioxidant | Spiked | T = 0 | T = 4 w | T = 8 w | T = 12 w |
| Type III | Vitamin E | No | 0.50 | 0.87 | 1.25 | NA |
| | Vitamin E | Yes | 0.50 | 2.21 | 4.12 | NA |
| Type III | BHT | No | 0.60 | 1.02 | 1.45 | NA |
| | BHT | Yes | 0.70 | 2.30 | 4.22 | NA |
| Type III | None | No | 0.60 | 0.94 | 1.40 | NA |
| | None | Yes | 0.60 | 2.28 | 4.25 | NA |
| Type IV | None | No | 0.50 | 0.48 | 0.64 | 0.50 |
| | None | Yes | 0.40 | 0.61 | 0.86 | 0.92 |

NA = Not assessed

### Example 5. Stability Testing of Type IV Formulations of Compound I in a Hardgel Capsule, 150 mg Strength

Stability data for hardgel capsules, 150 mg strength, prepared according to the method of Example 3, was obtained. Clear 7 mL glass vials with polypropylene screw cap containing the hardgel capsules of Example 3 were stored at ambient conditions (15-25′C in the dark). The assay and levels of two key impurities (COR176-6 and CORT125863) were determined after 12 months and 24 months.

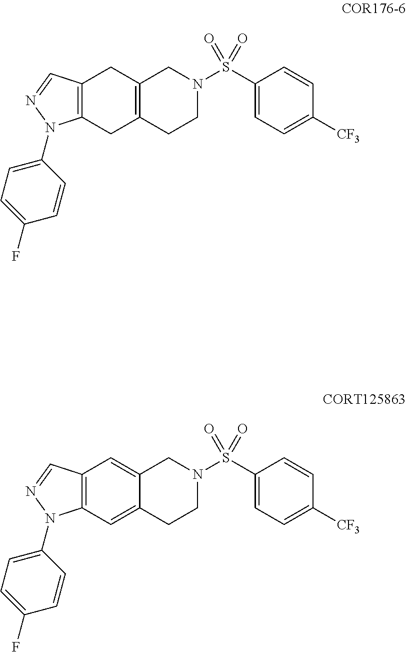 [SMILES: O=S(=O)(c1ccc(C(F)(F)F)cc1)N1CCC2=C(Cc3cnn(-c4ccc(F)cc4)c3C2)C1 ; O=S(=O)(c1ccc(C(F)(F)F)cc1)N1CCc2cc3c(cnn3-c3ccc(F)cc3)cc2C1]

TABLE 10
Stability Data for Type IV Formulations of
Compound I in a Hardgel Capsule, 150 mg (Glass Vials)

| Test Parameter | Initial | 12 months | 24 months |
|---|---|---|---|
| Compound I Assay (% of label claim) | 100.8 | 99.0 | 97.2 |
| COR176-6 (% area) | <0.05 | <0.05 | <0.05 |
| CORT125863 (% area) | 0.06 | 0.14 | 0.21 |
| Total Impurities (% area) | 0.5 | 0.63 | 0.77 |

No chromatographic response factor was applied for the impurities.

### Example 6. Preparation of Type IV Formulations of Compound I in a Hardgel Capsule, 75 mg Strength

A Type IV formulation in hardgel capsules, 75 mg strength, was prepared according to the following method:
Step 1: The Vitamin E TPGS was preheated to a temperature of 60-65° C. until molten in a suitable oven and 1943.5 g was dispensed into a suitable mixing vessel. While maintaining a temperature of 40-50° C. and stirring, polyethylene glycol (169.0 g) and Compound I (487.5 g) were added to the vessel and the contents mixed until a visually homogeneous solution was formed. The resultant solution (fill matrix) was then stored at 45-55° C. until encapsulation.
Step 2: The molten fill matrix (400 mg) was encapsulated into size 0 hard gelatin capsules.
Step 3: The capsules were banded using a gelatin solution and then dried and sealed.
Step 4: The capsules were packed into their final packaging configuration in bottles. Alternative packaging such as blisters are considered.

TABLE 11
Type IV Formulation of Compound I
in a Hardgel Capsule, 75 mg Strength

| | | | |
|---|---|---|---|
| | | mg per | |
| Component | % w/w | capsule | Function |

Capsule Fill Matrix

| | | | |
|---|---|---|---|
| Compound I | 18.75 | 75.0 | Active ingredient |
| Polyethylene Glycol 400 | 6.50 | 26.0 | Solubilizer & dispersion |
| | | | agent |
| Vitamin E TPGS | 74.75 | 299.0 | Solubilizer & surfactant |
| Total in capsule fill: | 100.0 | 400.0 | |

Capsule Shell

| | | |
|---|---|---|
| Opaque-white capsule | 1 unit | Capsule shell |
| shell size 0 | | |
| Gelatin banding¹ | q.s | |

¹Applied as an aqueous solution containing 21.43% w/w gelatin, 1.17% w/w polysorbate 80 and 0.87% w/w FD&C blue dye. Water removed during the drying process.

### Example 7. Stability Testing of Type IV Formulation of Compound I in a Hardgel Capsule, 75 mg Strength

60 ml HPDE bottles containing 30 hardgel capsules of Example 5 were stored under ICH stability conditions. The assay and levels of the two key impurities (COR176-6 and CORT125863) were determined for up to 3 months.

TABLE 12
Room Temperature Storage (25° C./60% RH) of
Type IV Formulations of Compound I in
Hardgel Capsules, 75 mg Strength (HDPE Bottles)

| Test Parameter | Initial | 3 months |
|---|---|---|
| Compound I Assay | 98.4 | 103.1 |
| (% of label claim) | | |
| COR176-6 (% w/w) | <0.05 | <0.05 |
| CORT125863 (% w/w) | 0.14 | 0.18 |
| Total Impurities (% area) | 0.59 | 0.73 |

A chromatographic relative response factor (RRF) of 2.72 was applied to the COR176-6 impurity and an RRF of 1.30 applied to CORT125863.

TABLE 13
Accelerated Temperature Storage (40° C./75% RH)
of Type IV Formulations of Compound I in Hardgel
Capsules, 75 mg Strength (HDPE Bottles)

| Test Parameter | Initial | 1 month | 2 months | 3 months |
|---|---|---|---|---|
| Compound I Assay | 98.4 | 99.3 | 96.7 | 97.8 |
| (% of label claim) | | | | |
| COR176-6 (% w/w) | <0.05 | <0.05 | 0.08 | 0.15 |
| CORT125863 (% w/w) | 0.14 | 0.21 | 0.27 | 0.35 |
| Total Impurities (% area) | 0.59 | 0.84 | 1.10 | 1.20 |

A chromatographic relative response factor (RRF) of 2.72 was applied to the COR176-6 impurity and an RRF of 1.30 applied to CORT125863.

### Example 8. Preparation of Type IV Formulations of Compound I in a Softgel Capsule, 75 mg Strength

Type IV formulations in softgel capsules were prepared according to the following method, and in accordance with Table 1:
Step 1: The Vitamin E TPGS was preheated to a temperature of 65° C. until molten in a suitable oven and 747.5 g dispensed into a suitable mixing container. While maintaining a temperature of 40° C. and stirring, polyethylene glycol (65.0 g) and Compound I (187.5 g) were added to the container and the contents mixed until a visually homogeneous solution was formed. The resultant solution (fill matrix) was then deaerated, blanketed with nitrogen and stored at 40-45° C. until encapsulation.
Step 2: The gel mass was prepared using gelatin, purified water, and sorbitol special glycerin blend. The gel mass was then color converted by adding titanium dioxide and red iron oxide.
Step 3: The fill matrix (400 mg) was encapsulated into softgel capsules.
Step 4: The capsules were dried and hardened in tumble dryers and drying chambers.
Step 5: Bulk capsules were packaged in polyethylene-lined corrugated shipping cartons.
Step 6: The capsules were packed into their final packaging configuration in bottles or blisters.

### Example 9. Stability of Type IV Formulations of Compound I in a Softgel Capsule, 75 mg Strength

Blisters containing 75 mg strength softgel capsules as provided in Example 8 were stored under ICH conditions. The assay and levels of the two key impurities (COR176-6 and CORT125863) were determined for up to 22 months at 25° C. and 60% relative humidity (RH), as shown in Table 14, and for up to 6 months at 40° C. and 75% RH as shown in Table 15.

TABLE 14
Room Temperature Storage (25° C./60% RH) Softgel Capsules 75 mg in Blisters

| Test | Timepoint (months) | | | | | | | |
|---|---|---|---|---|---|---|---|---|
| Parameter | Initial | 1 | 2 | 3 | 6 | 12 | 18 | 22 |
| Compound I | 104.3 | 103.5 | 104.8 | 105.4 | 104.1 | 107.0 | 102.3 | 103.1 |
| Assay (% of | | | | | | | | |
| label claim) | | | | | | | | |
| COR176-6 | ND | ND | 0.11 | 0.15 | 0.27 | 0.49 | 0.69 | 0.85 |
| (% w/w) | | | | | | | | |
| CORT125863 | 0.09 | 0.12 | 0.13 | 0.15 | 0.19 | 0.28 | 0.39 | 0.44 |
| (% w/w) | | | | | | | | |
| Total | 0.46 | 0.49 | 0.60 | 0.64 | 0.88 | 1.25 | 1.66 | 2.08 |
| Impurities | | | | | | | | |
| (% area) | | | | | | | | |

A chromatographic relative response factor has been applied to COR176-6 (2.72) and CORT125862 (1.30).

TABLE 15
Accelerated Temperature Storage (40° C./75% RH)
Softgel Capsules 75 mg Strength in Blisters

| | | 1 | 2 | 3 | 6 |
|---|---|---|---|---|---|
| Test Parameter | Initial | month | months | months | months |
| Compound I Assay | 104.3 | 101.9 | 103.6 | 102.2 | 100.7 |
| (% of label claim) | | | | | |
| COR176-6 (% w/w) | ND | 0.32 | 0.65 | 0.98 | 1.93 |
| CORT125863 (% w/w) | 0.09 | 0.22 | 0.33 | 0.44 | 0.72 |
| Total Impurities (% area) | 0.46 | 0.99 | 1.45 | 1.94 | 3.33 |

A chromatographic relative response factor has been applied to COR176-6 (2.72) and CORT125863 (1.30).

Although the foregoing invention has been described in some detail by way of illustration and example for purposes of clarity of understanding, one of skill in the art will appreciate that certain changes and modifications may be practiced within the scope of the appended claims. In addition, each reference, including all of the U.S. patents, U.S. patent application publications, U.S. patent applications, foreign patents, foreign patent applications and non-patent publications referred to in this specification are incorporated herein by reference, in their entirety, to the extent not inconsistent with the present description. Where a conflict exists between the instant application and a reference provided herein, the instant application shall dominate.

What is claimed is:

1. A composition comprising:
Compound I, (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone: 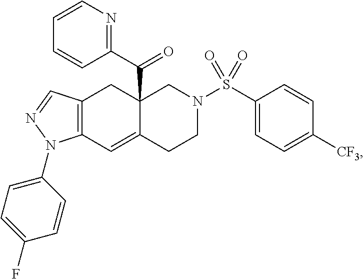 [SMILES: O=C(c1ccccn1)[C@]12Cc3cnn(-c4ccc(F)cc4)c3C=C1CCN(S(=O)(=O)c1ccc(C(F)(F)F)cc1)C2]
in an amount of from 1 to 25% (w/w);
a cosolvent, in an amount of from 1 to 25% (w/w); and
a surfactant, in an amount of from 50 to 90% (w/w).

2. The composition of claim 1, wherein Compound I is present in an amount of from 15 to 20% (w/w).

3. The composition of claim 1 or 2, wherein Compound I is present in an amount of from 18 to 20% (w/w).

4. The composition of any one of claims 1 to 3, wherein Compound I is present in an amount of about 18.75% (w/w).

5. The composition of any one of claims 1 to 4, wherein Compound I is present in an amount of from 50 to 150 mg.

6. The composition of any one of claims 1 to 5, wherein Compound I is present in an amount of about 75 mg.

7. The composition of any one of claims 1 to 5, wherein Compound I is present in an amount of about 150 mg.

8. The composition of any one of claims 1 to 7, wherein the cosolvent comprises polyethylene glycols (PEG), ethanol, propylene glycol, glycerol, diethylene glycol monoethyl ether, glycofurol, triacetin, N-methyl-2-pyrrolidone, dimethylacetamide, dimethylsulfoxide, propylene glycol esters, or combinations thereof.

9. The composition of any one of claims 1 to 8, wherein the cosolvent comprises polyethylene glycol (PEG).

10. The composition of any one of claims 1 to 9, wherein the cosolvent comprises polyethylene glycol having a molecular weight of about 400 Daltons (PEG400).

11. The composition of any one of claims 1 to 10, wherein the cosolvent comprises PEG400 in an amount of from 5 to 8% (w/w).

12. The composition of any one of claims 1 to 11, wherein the cosolvent comprises PEG400 in an amount of from 6 to 7% (w/w).

13. The composition of any one of claims 1 to 12, wherein the cosolvent comprises PEG400 in an amount of about 6.5% (w/w).

14. The composition of any one of claims 1 to 13, wherein the surfactant comprises Vitamin E polyethylene glycol succinate (Vitamin E TPGS), sorbitan monooleate, polysorbate, Solutol HS 15, poloxamer 407, Labrafil® M-1944CS, Labrafil® M-2125CS, Labrasol®, Softigen® 767, Gelucire, Kolliphor, or combinations thereof.

15. The composition of any one of claims 1 to 14, wherein the surfactant comprises Vitamin E polyethylene glycol succinate (Vitamin E TPGS).

16. The composition of any one of claims 1 to 15, wherein the surfactant comprises Vitamin E polyethylene glycol succinate (Vitamin E TPGS) in an amount of from 70 to 80% (w/w).

17. The composition of any one of claims 1 to 16, wherein the surfactant comprises Vitamin E polyethylene glycol succinate (Vitamin E TPGS) in an amount of from 74 to 76% (w/w).

18. The composition of any one of claims 1 to 17, wherein the surfactant comprises Vitamin E polyethylene glycol succinate (Vitamin E TPGS) in an amount of about 74.75% (w/w).

19. The composition of any one of claims 1 to 18, comprising:
Compound I, in an amount of about 18.75% (w/w);
PEG400, in an amount of about 6.5% (w/w); and
Vitamin E TPGS, in an amount of about 74.75% (w/w).

20. The composition of any one of claims 1 to 19, comprising:
Compound I, in an amount of about 75 mg;
PEG400, in an amount of about 26 mg; and
Vitamin E TPGS, in an amount of about 299 mg.

21. The composition of any one of claims 1 to 20, consisting of:
Compound I, in an amount of 75 mg;
PEG400, in an amount of 26 mg; and
Vitamin E TPGS, in an amount of 299 mg.

22. The composition of any one of claims 1 to 19, comprising:
Compound I, in an amount of about 50 mg;
PEG400, in an amount of about 17 mg; and
Vitamin E TPGS, in an amount of about 199 mg.

23. The composition of any one of claims 1 to 19, comprising:
Compound I, in an amount of about 100 mg;
PEG400, in an amount of about 35 mg; and
Vitamin E TPGS, in an amount of about 399 mg.

24. The composition of any one of claims 1 to 19, comprising:
Compound I, in an amount of about 150 mg;
PEG400, in an amount of about 52 mg; and
Vitamin E TPGS, in an amount of about 598 mg.

25. The composition of any one of claims 1 to 18, comprising
Compound I, in an amount of about 15% (w/w);
PEG400, in an amount of about 6.5% (w/w); and
Vitamin E TPGS, in an amount of about 78.5% (w/w).

26. The composition of any one of claims 1 to 25, comprising:
Compound I, in an amount of about 60 mg;
PEG400, in an amount of about 26 mg; and
Vitamin E TPGS, in an amount of about 314 mg.

27. The composition of any one of claims 1 to 18, comprising
Compound I, in an amount of about 20.0% (w/w);
PEG400, in an amount of about 6.5% (w/w); and
Vitamin E TPGS, in an amount of about 73.5% (w/w).

28. The composition of any one of claims 1 to 27, comprising:
Compound I, in an amount of about 80 mg;
PEG400, in an amount of about 26 mg; and
Vitamin E TPGS, in an amount of about 294 mg.

29. The composition of any one of claims 1 to 21 or 25 to 28, in a unit dosage form of 400 mg.

30. A unit dosage form for oral administration, consisting essentially of a capsule containing a composition of any one of claims 1 to 29.

31. The unit dosage form of claim 30, wherein the capsule is a softgel capsule or a hardgel capsule.

32. The unit dosage form of claim 30 or 31, consisting essentially of a softgel capsule containing the composition consisting of:
Compound I, in an amount of 75 mg;
polyethylene glycol having a molecular weight of about 400 Daltons (PEG400), in an amount of 26 mg; and
Vitamin E polyethylene glycol succinate (Vitamin E TPGS), in an amount of 299 mg.

33. The unit dosage form of claim 30, wherein the capsule is a hardgel capsule.

34. The unit dosage form of claim 30 or 33, consisting essentially of a hardgel capsule containing the composition consisting of:
Compound I, in an amount of 75 mg;
polyethylene glycol having a molecular weight of about 400 Daltons (PEG400), in an amount of 26 mg; and
Vitamin E polyethylene glycol succinate (Vitamin E TPGS), in an amount of 299 mg.

35. The unit dosage form of claim 30 or 33, consisting essentially of a hardgel capsule containing the composition consisting of:
Compound I, in an amount of 50 mg;
polyethylene glycol having a molecular weight of about 400 Daltons (PEG400), in an amount of 17 mg; and
Vitamin E polyethylene glycol succinate (Vitamin E TPGS), in an amount of 199 mg.

36. The unit dosage form of claim 30 or 33, consisting essentially of a hardgel capsule containing the composition consisting of:
Compound I, in an amount of 100 mg;
polyethylene glycol having a molecular weight of about 400 Daltons (PEG400), in an amount of 35 mg; and
Vitamin E polyethylene glycol succinate (Vitamin E TPGS), in an amount of 399 mg.

37. The unit dosage form of claim 30 or 33, consisting essentially of a hardgel capsule containing the composition consisting of:
Compound I, in an amount of 150 mg;
polyethylene glycol having a molecular weight of about 400 Daltons (PEG400), in an amount of 52 mg; and
Vitamin E polyethylene glycol succinate (Vitamin E TPGS), in an amount of 598 mg.

38. A method of treating a disorder or condition through modulating a glucocorticoid receptor, comprising administering to a subject in need of such treatment, a therapeutically effective amount of a composition of any one of claims 1 to 29, or a unit dosage form of any one of claims 30 to 37, thereby treating the disorder or condition.

39. A method of treating a disorder or condition through antagonizing a glucocorticoid receptor, comprising administering to a subject in need of such treatment, a therapeutically effective amount of a composition of any one of claims 1 to 29, or a unit dosage form of any one of claims 30 to 37, thereby treating the disorder or condition.

40. A method of treating amyotrophic lateral sclerosis (ALS), comprising administering to a subject in need thereof, a therapeutically effective amount of a composition of any one of claims 1 to 29, or a unit dosage form of any one of claims 30 to 37, thereby treating ALS.

41. A method of treating Alzheimer's disease, comprising administering to a subject in need thereof, a therapeutically effective amount of a composition of any one of claims 1 to 29, or a unit dosage form of any one of claims 30 to 37, thereby treating Alzheimer's.

42. A method of treating Huntington's disease, comprising administering to a subject in need thereof, a therapeutically effective amount of a composition of any one of claims 1 to 29, or a unit dosage form of any one of claims 30 to 37, thereby treating Huntington's.
